(19) United States
(12) Patent Application Publication
(10) Pub. No.: US 2004/0049257 A1
(43) Pub. Date: Mar. 11, 2004

(54) Covered expandable stent

(76) Inventors: Jon Harald Kaspersen, Trondheim (NO); Jan Lundbom, Trondheim (NO)

Correspondence Address: WENDEROTH, LIND & PONACK, L.L.P., 2033 K STREET N. W., SUITE 800, WASHINGTON, DC, 20006-1021, US

(21) Appl. No.: 10/363,396

(22) PCT Filed: Sep. 4, 2001
(86) PCT No.: PCT/NO01/00365

(30) Foreign Application Priority Data
Sep. 5, 2000 (NO) 20004420

(51) Int. Cl. A61F 2/06

(52) U.S. Cl. 623/1.13; 623/1.35

(57) ABSTRACT

Covered expandable stent (1) having preferably one or several side branches (2) for inserting into the vascular system by means of an introducer sleeve (11). The covered expandle stent (1) comprises:—a tubular body (4) provided with sutures (5) running along and fixed at spots on the circumference of the inner surface at a number ok spaced apart locations in the lengthwise direction of the stent (1), which sutures (5) in a compressed state of the stent form loops extending to the centre of the stent (1),—guide wire(s) (6,7) inserted through the loops keeping the stent (1) in a first compressed state, prior to operation the stent (1) having been further compressed and inserted into the introducer sleeve (1), whereby during operation the stent (1) is pushed out of the introducer sleeve (11) assuming the first compressed state in which the stent (1) is navigable so as to achieve the correct location, the guide wire (6,7) being adapted to be removed thereafter, whereby the stent (1) assumes a fully expanded state in which the loops of the sutures (5) extends around the circumference of the inner surface of the tubular body (4).

### FIELD OF THE INVENTION

The present invention relates to a covered expandable stent having preferably one or several side branches for inserting into the vascular system by means of an introducer sleeve.

It is also of great importance that the graft to be deployed is customised to the patient's anatomy.

### BACKGROUND OF THE INVENTION

Stent grafts are at present well known in medical practice. They usually have a cylindrical shape, and are often covered with biological compatible material such as Polyester or ePTFE. Several kinds of stents exists, some are expandable due to the rise in temperature when they are exposed to the blood, some are preloaded “springs” and some have to be expanded by a balloon.

Covered stent grafts (or prosthesis) are devices used to treat aneurysms. These prosthesis are inserted into the vascular anatomy system to exclude the aneurysm from the blood stream. Thus, insertion of a vascular prosthesis from the iliac arteries (see FIG. 1a) can treat abdominal aortic aneurysms. The prosthesis is preloaded in a tube (or introducer), and when correctly placed the prosthesis is pushed out of the tube (introducer). It is vital that the prosthesis is placed correctly. It is especially important to keep the renal arteries open (see FIG. 1a). On the other hand the prosthesis needs to be firmly connected to the artery (in this case the Aorta). This means that there needs to be a healthy area between the start of the aneurysm (proximal end) and the renal arteries. This limitation in use applies to other parts of the vascular system as well (Aortic arc, Iliac etc.).

### SUMMARY OF THE INVENTION

The present invention solves the limitation in use by providing a customised covered expandable stent, having one or several side branches for inserting into the vascular system by means of an introducer sleeve, characterized by comprising:

a tubular body provided with sutures running along and fixed at spots on the circumference of the inner surface at a number of spaced apart locations in the lengthwise direction of the stent, which sutures in a compressed state of the stent form loops extending to the centre of the stent,

a guide wire(s) inserted through the loops keeping the stent in a first compressed state, prior to operation the stent having been further compressed and inserted into the introducer sleeve, whereby during operation the stent is pushed out of the introducer sleeve assuming the first compressed state in which the stent is navigable so as to achieve the correct location, the guide wire being adapted to be removed thereafter, whereby the stent assumes a fully expanded state in which the loops of the sutures extends around the circumference of the inner surface of the tubular body.

Preferred embodiments of the covered expandable stent according to claim 1 are further explained in claims 2-6.

Other objects and advantages of the invention will appear from the following description.

### BRIEF DESCRIPTION OF THE DRAWINGS

For a detailed description of a preferred embodiment of the invention, reference will now be made to the accompanying drawings, wherein:

FIG. 1a is a sketch showing a stent graft located in the vascular system,

FIG. 1b is a sketch showing a 3D image of the vascular anatomy of a patient,

FIG. 2a shows a stent according to the invention in a first compressed state including a guide wire,

FIG. 2b shows the stent in a fully expanded state with the guide wire removed,

FIG. 3a is a picture of a stent with guide wire mounted,

FIG. 3b is a picture of the stent of FIG. 3a with the guide wire removed,

FIGS. 4-9 are sketches showing the different steps of deploying the graft in the vascular system, and

FIG. 10 is a sketch showing an arrangement for positioning the stent in the vascular system.

### DETAILED DESCRIPTION OF THE PREFERRED EMBODIMENT

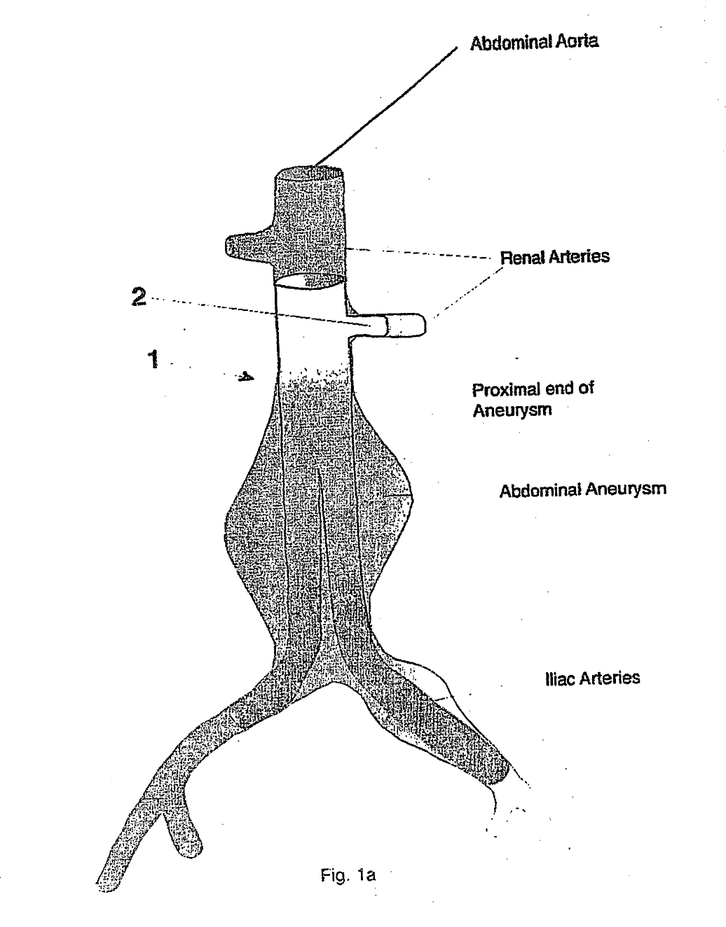

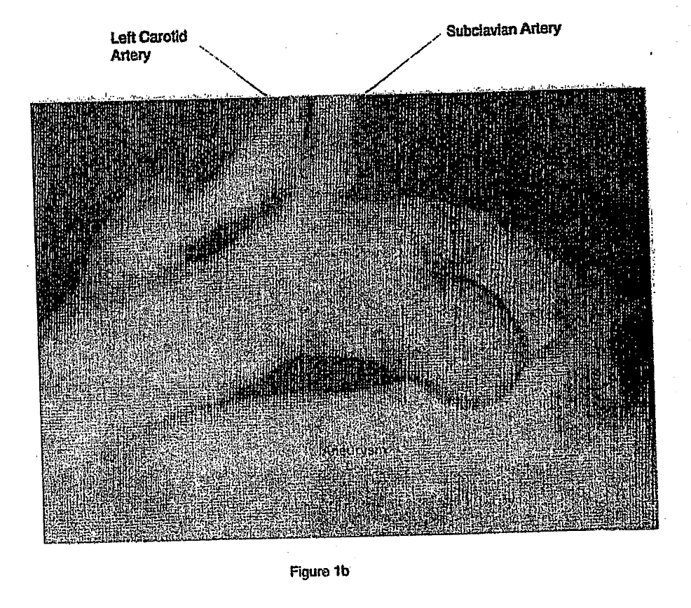

With referance to FIG. 1a a bifurcated stent-graft or a covered expandable stent 1 having one side branch 2 is shown inserted into the vascular system to exclude the aneurysm from the blood stream.

First of all it is essential that the stent-graph (prosthesis) 1 to be deployed is customised according to the patient's anatomy. The shape and dimensions of the prosthesis must be individually adapted. To make a customised vascular prosthesis three-dimensional (3D) information of the actual vascular system is needed. This can be obtained by several imaging techniques. Magnetic Resonant (MR) or Computed Tomography (CT) has been used, but another alternative can be 3D IntraVascular UltraSound (IVUS). The latter technique is described in U.S. Pat. No. 5,830,145, “Enhanced accuracy of three-dimensional intraluminal ultrasound (ILUS) image reconstruction”. With the three-dimensional information available, a customised covered bifurcated stent graft 1 with a side branche 2 can be manufactured. All dimensions like diameter, length, angles, curvature etc. can be decided from the 3D image available. An example of such an image is shown in FIG. 1b.

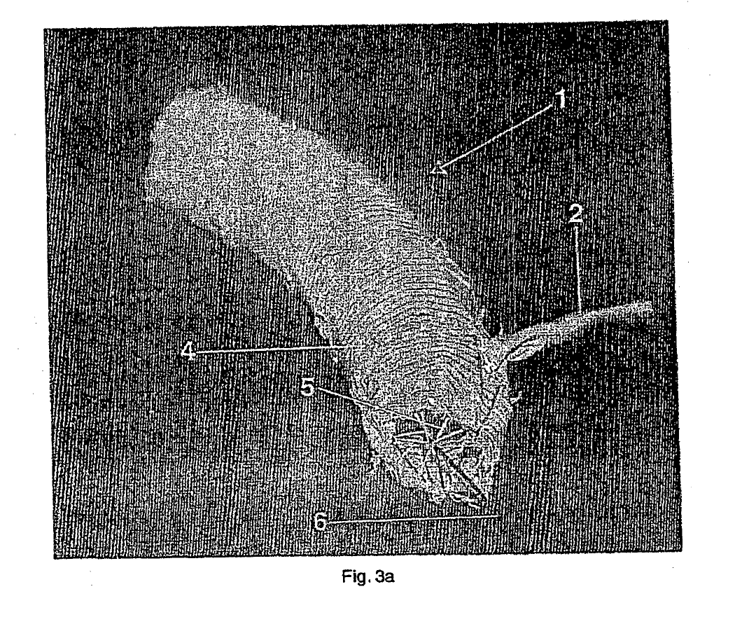

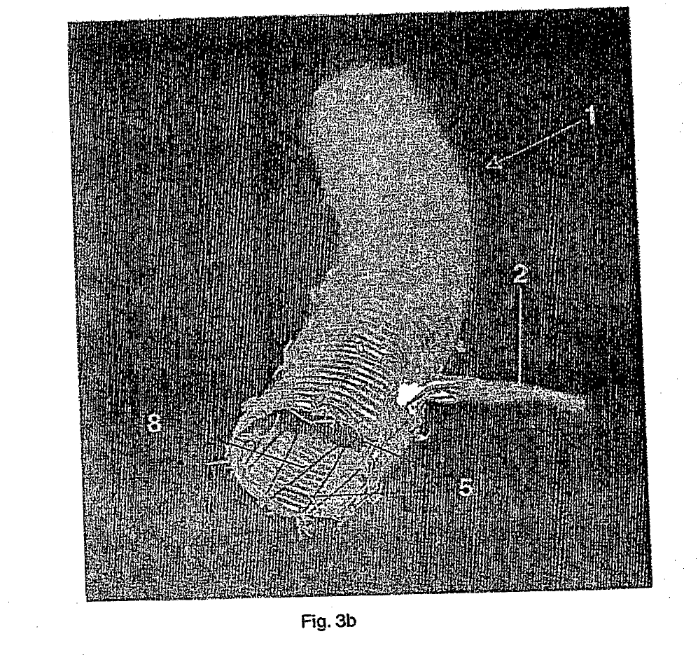

The customised graft 1 has to be securely deployed. In order to do that this invention describes a unique, but simple method to ensure that the graft 1 is placed correctly. The deployment of the graft 1 in the vascular system will be explained below with references to FIGS. 4-9. First of all the graft 1 has to be loaded into an introducer 11 with guide-wire 6,7 mounted. The guide-wire 6,7 is mounted in such a way that the graft 1 will not expand fully when it is pushed out of the introducer 11. This will enable navigation of the graft 1 also after it has been released from the introducer 11. In order to successfully deploy a graft 1 with a side branch 2 it is essential to be able to navigate the graft 1 at this stage of the procedure. This is the main object of this invention. A picture of the guide wire 6 mounted is shown in FIG. 3a. Suture 5 is attached around the graft circumferencially in the way shown in FIG. 3b. This suture 5 is a certain percentage longer than the circumference of the graft 1. A “short” suture keeps the graft 1 more compact than a “long” suture. Another object with this invention is that when the graft is mounted on the guide wire 6,7 it is possible to move it in the long-axis of the graft 1 by pushing with an “over-the-wire” catheter or similar instrument.

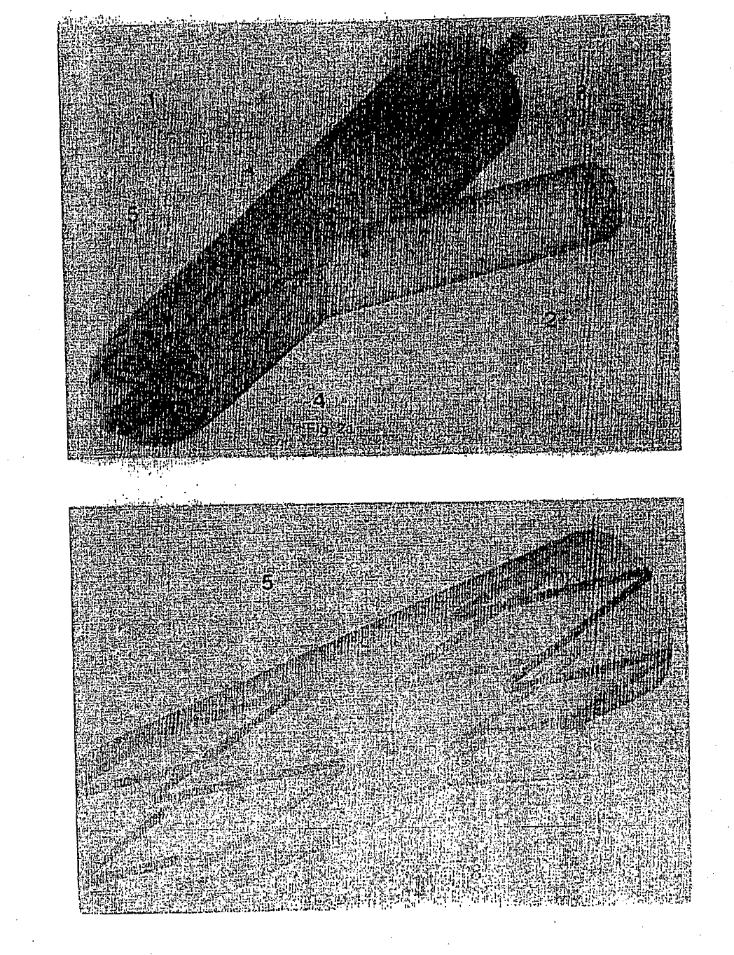

The body of the stent graft consists of any kind of self-expandable stent, such as a GienturcoZ-stent. The GienturcoZ-stent includes self-expandable wires 8 made of stainless steel, see FIG. 2b, arranged in a zigzag pattern on the circumference of the inner surface of the stent 1.

Again with reference to FIGS. 4-9 the procedure of deploying a graft 1 with a side branch 2 will be further described. First of all, guide-wires 6,7 must be inserted both into the main vessel and into the branching vessel. FIG. 4 shows the guide-wires 6,7 after they have been inserted.

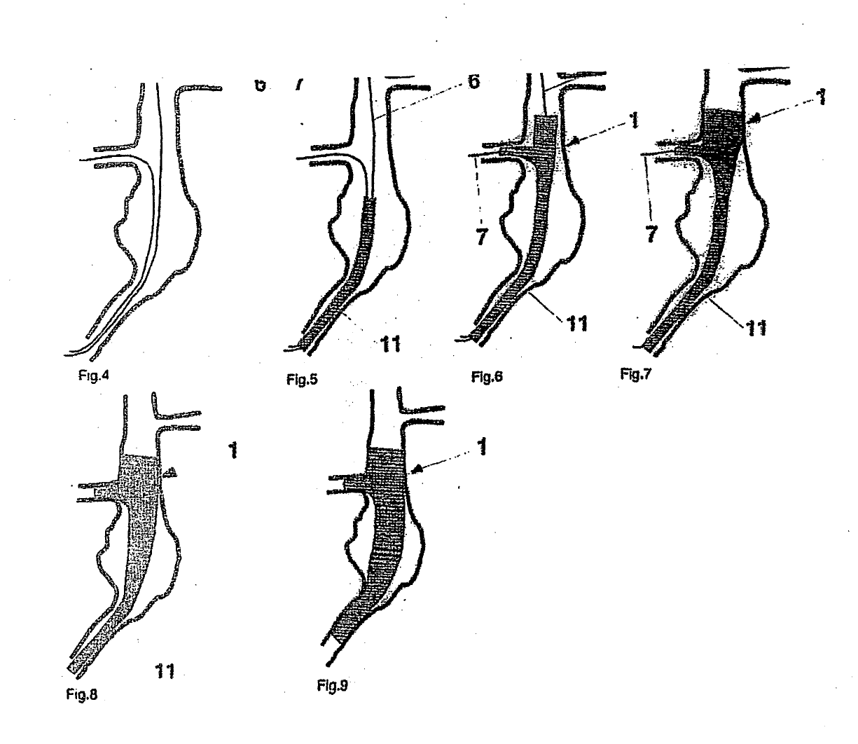

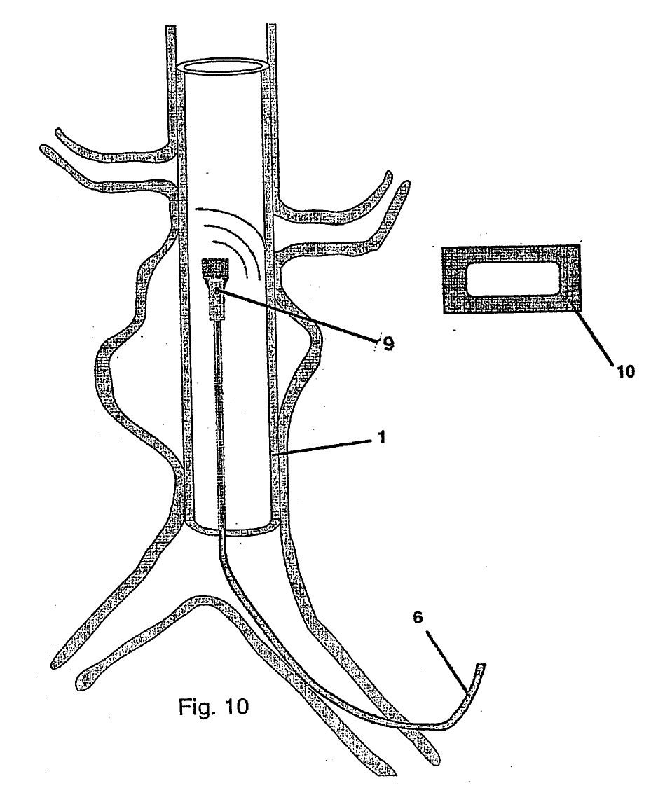

In FIG. 5 the introducer 11 and the guide-wires 6,7 are shown. The graft 1 is still kept inside the introducer 11 with one guide-wire 6 through the main part of the graft 1 and one guide-wire 7 through the branching part of the graft 1. When the graft 1 is pushed out of the introducer 11, as shown in FIG. 6. The main part of the graft 1 will follow the path of the guide-wire 6, which is passing through it, and the branching part of the graft 1 will follow the path of the guide-wire 7 passing through the side branch of the graft 1 and into the branching vessel. Since the graft 1 is mounted on the guide-wires as shown in FIG. 3a, it is still not attached to the vessel wall. This unique feature enables the surgeon to move and/or rotate the graft 1 until it is found to be placed correctly, shown in FIG. 7. Once the graft 1 is correctly placed, the guide-wire 6 through the main part is removed resulting in a sudden expansion of the graft 1 main part. The main part of the graft 1 is now fully expanded and therefore also attached to the vessel wall, shown in FIG. 7. Next, the guide-wire 7 through the branching vessel is removed and in the same way as the main part, the branching part is expanded and attached to the vessel wall, shown in FIG. 8. (E.g. The side branch should be fixated with a stent or the side branch should consist of a stent graft, self-expanding or balloon-expandable.) Another possibility is to expand the branching part of the graft 1 first, which enables correctional navigation of the main part after the branching part has been attached to the vessel wall. Removing the introducer 11, as shown in FIG. 9 completes the procedure. The lower part of the graft 1 will expand and attach to the vessel wall immediately after it has left the introducer 11.

A micro positioning device 9 and/or the introducer 11 may be mounted on the guidewire(s) 6,7 and/or the stent graft 1. It will then be possible to mark the position of the stent graft 1 and/or the guidewires 6,7 on a position receiving device 10 i.e. in a 2D and/or 3D visualisation scene and thereby ensure a correct deployment fixation. The visualisation scene may be generated from 3D preoperative MR and/or CT or from intraoperative ultrasound.

1. Covered expandable stent (1) having preferably one or several side branches (2) for inserting into the vascular system by means of an introducer sleeve (11), as the stent comprises a tubular body (4) provided with sutures (5) running along and fixed at spots on the circumference of the inner surface at a number of spaced apart locations in the lengthwise direction of the stent (1), characterized in that the sutures (5) in a compressed state of the stent form loops extending to the centre of the stent (1), whereby a combined guide and release wire(s) (6,7) are inserted through the internal loops keeping the stent (1) in a first compressed state, prior to operation the stent (1) having been further compressed and inserted into the introducer sleeve (11), whereby during operation the stent (1) is pushed out of the introducer sleeve (11) assuming the first compressed state in which the stent (1) is navigable so as to achieve the correct location, whereafter the combined guide and the release wires (6,7) is (are) removed and the stent (1) assumes a fully expanded state in which the loops of the sutures (5) extends around the circumference of the inner surface of the tubular body (4).

2. Covered expandable stent (1) according to claim 1, characterized by the fully expanded state of the stent (1) is provided by self-expandable wires (8) fixed and arranged in a zigzag pattern on the circumference of the inner surface of the stent (1).

3. Covered expandable stent (1) according to claim 1 or 2, characterized by the self-expandable wires (8) are formed of stainless steel.

4. Covered expandable stent (1) according to claim 1,2 or 3, characterized by the guidwire(s) (6,7) is (are) provided with a micro positioning device (9).

5. Covered expandable stent (1) according to anyone of the previous claims, characterized by further including a micro positioning device (10) mounted on the stent (1).

6. Covered expandable stent (1) according to claims 4 or 5, characterized by the micro positioning device (9) is (are) communicating with an external position receiving device (10).
